(19) United States
(12) Patent Application Publication
(10) Pub. No.: US 2012/0121471 A1
(43) Pub. Date: May 17, 2012

(54) Air filtration media comprising metal-doped silicon-based gel materials with pre-reduced oxidizing agents

(76) Inventors: Terry W. Nassivera, Bel Air, MD (US); Michael C. Withiam, Landenberg, PA (US); Fitzgerald A. Sinclair, Bear, DE (US); Ruth G. Heffes, Glen Burnie, MD (US)

(21) Appl. No.: 11/823,265

(22) Filed: Jun. 27, 2007

(51) Int. Cl. A61L 9/00

(52) U.S. Cl. 422/122

(57) ABSTRACT

The present invention relates generally to an environmental control unit for use in air handling systems that provides highly effective filtration of noxious gases (such as ammonia). Such a filtration system utilizes novel metal-doped silica-based gels to trap and remove such undesirable gases from an enclosed environment. Such gels exhibit specific porosity requirements and density measurements. Furthermore, in order for proper metal doping to take effect, such gels must be treated while in a wet state. The combination of these particular properties and metal dopant permits highly effective noxious gas filtration such that uptake and breakthrough results are attained, particularly in comparison with prior silica gel filtration products. Also included is the presence of an oxidizing agent (either in reduced or pre-deuced form) to aid in capturing nitrogen dioxide and preventing conversion of such a product to NO. Methods of using and specific filter apparatuses are also encompassed within this invention.

### FIELD OF THE INVENTION

The present invention relates generally to an environmental control for use in air handling systems that provides highly effective filtration of noxious gases (such as ammonia). Such a filtration system utilizes novel metal-doped silica-based gels to trap and remove such undesirable gases from an enclosed environment. Such gels exhibit specific porosity requirements and density measurements. Furthermore, in order for the most effective metal doping to take effect, such gels are preferably treated with a multivalent metal salt while in a wet state. The combination of these particular properties and metal dopants, permit highly effective noxious gas filtration such that excellent uptake and breakthrough results are attained, particularly in comparison with prior media filtration products. Also included is the presence of an oxidizing agent (either in reduced or pre-reduced form) to aid in capturing nitrogen oxides and preventing conversion of such a product to NO. Methods of using and specific filter apparatuses are also encompassed within this invention.

### BACKGROUND OF THE INVENTION

There is an ever-increasing need for air handling systems that include air filtration systems that can protect an enclosure against noxious airborne vapors and particulates released in the vicinity of the enclosure. Every year there are numerous incidents of noxious vapors contaminating building environments and causing illness and disruptions. There is also a current effort to protect buildings and other significant enclosures against toxic airborne vapors and particulates being released as part of terrorist acts. As a result, new filter design requirements have been promoted by the military to protect from certain toxic gases. Generally speaking, whether in a civilian or military setting, a typical air filtration system that contains only a particulate filter (for example, a cardboard framed fiberglass matt filter) provides no protection at all against toxic vapors. Commercially available electrostatic fiber filters exhibit higher removal efficiencies for smaller particles than standard dust filters, but they have no vapor filtration capability. HEPA (“High-Efficiency Particulate Air”) filters are used for high-efficiency filtration of airborne dispersions of ultrafine solid and liquid particulates such as dust and pollen, radioactive particle contaminants, and aerosols. However, where the threat is a gaseous chemical compound or a gaseous particle of extremely small size (i.e., <0.001 microns), the conventional commercially-available HEPA filters cannot intercept and control those types of airborne agents.

The most commonly used filter technology to remove vapors and gases from contaminated air is activated carbon. Such carbon-based gas filtration has been implemented in a wide variety of vapor-phase filtration applications including gas masks and military vehicle and shelter protection. In these applications, activated carbon impregnated with metal salts is used to remove a full range of toxic vapors (such as arsine, Sarin gas, etc.). These toxic gases require a high filtration efficiency typically not needed for most commercial applications. To the contrary, typical commercial filters generally include activated carbon materials on or incorporated within non-woven fabrics (fiber mats, for instance), with coexisting large fixed beds of packed adsorbent particles. Such commercial filters used for air purification generally are used until an easily measurable percentage (e.g., 10%) of the challenge chemical(s) concentration is measured in the effluent. Greater long-term efficiency is desired for gas masks and/or military vehicle applications.

Impregnated, activated carbons are used in applications where required to remove gases that would not otherwise be removed through the use of unimpregnated activated carbons. Such prior art impregnated carbon formulations often contain copper, chromium and silver impregnated on an activated carbon. These adsorbents are effective in removing a large number of toxic materials, such as cyanide-based gases and vapors.

In addition to a number of other inorganic materials, which have been impregnated on activated carbon, various organic impregnates have been found useful in military applications for the removal of cyanogen chloride. Examples of these include triethylenediamine (TEDA) and pyridine-4-carboxylic acid.

Various types of high-efficiency filter systems, both commercial and military types, have been proposed for building protection using copper-silver-zinc-molybdenum-triethylenediamine impregnated carbon for filtering a broad range of toxic chemical vapors and gases. However, such specific carbon-based filters have proven ineffective for other gases, such as, ammonia, ethylene oxide, formaldehyde, and nitrogen oxides. As these gases are quite prominent in industry and can be harmful to humans when present in sufficient amounts (particularly within enclosed spaces), and, to date, other filter devices have proven unsuitable for environmental treatment and/or removal thereof, there exists a definite need for a filter mechanism to remedy these deficiencies, particularly in both high and low relative humidity (RH) environments. Each chemical is affected differently by adsorbed water. For ammonia, it is most difficult (design limiting) to filter at a low relative humidity since adsorbed water actually enhances the ammonia affinity of the target adsorbents. For ethylene oxide the reverse is true since exposure to high humidity is problematic in designing a proper filter system. To date, no filtration system having a relatively small amount of filter medium present has been provided that effectively removes such gases at their design limiting RH for long durations of time at relatively high challenge concentrations (e.g., 1,000 ppm) without eventually eluting through the filter.

It has been realized that silica-based compositions make excellent gas filter media. However, little has been provided within the pertinent prior art that concerns the ability to provide uptake and breakthrough levels by such filter media on a permanent basis and at levels that are acceptable for long-term usage. Uptake basically is a measure of the ability of the filter medium to capture a certain volume of the subject gas; breakthrough is an indication of the saturation point for the filter medium in terms of capture. Thus, it is highly desirable to find a proper filter medium that exhibits a high uptake (and thus quick capture of large amounts of noxious gases) and long breakthrough times (and thus, coupled with uptake, the ability to not only effectuate quick capture but also extensive lengths of time to reach saturation). The standard filters in use today are limited for noxious gases, such as ammonia and nitrogen dioxide (NO₂), to slow uptake and relatively quick breakthrough times. There is a need to develop a new filter medium that increases uptake and breakthrough, as a result.

The closest art concerning the removal of gases such as ammonia utilizing a potential silica-based compound doped with a metal is taught within WO 00/40324 to Kemira Agro Oy. Such a system, however, is primarily concerned with providing a filter media that permits regeneration of the collected gases, presumably for further utilization, rather than permanent removal from the atmosphere. Such an ability to easily regenerate (i.e., permit release of captured gases) such toxic gases through increases of temperature or changes in pressure unfortunately presents a risk to the subject environment. To the contrary, an advantage of a system as now proposed is to provide effective long-duration breakthrough (thus indicating thorough and effective removal of unwanted gases in substantially their entirety from a subject space over time, as well as thorough and effective uptake of substantially all such gases as indicated by an uptake measurement. The Kemira reference also is concerned specifically with providing a dry mixture of silica and metal (in particular copper I salts, ultimately), which, as noted within the reference, provides the effective uptake and regenerative capacity sought rather than permanent and effective gas (such as ammonia) removal from the subject environment. The details of the inventive filter media are discussed in greater depth below.

### BRIEF DESCRIPTION OF THE INVENTION

According to one aspect of this invention, a filter medium comprising multivalent metal-doped silicon-based gel materials, wherein said materials exhibit a BET surface area of between than 100 and 600 m²/g (preferably 100 to 300); a pore volume of between about 0.18 cc/g to about 0.7 cc/g as measured by nitrogen porosimetry; a cumulative surface area measured for all pores having a size between 20 and 40 Å of between 50 and 150 m²/g; and wherein the multivalent metal doped on and within said silicon-based gel materials is present in an amount of from 5 to 25% by weight of the total amount of the silicon-based gel materials. Preferably, the filter medium exhibits a BET surface area is between 150 m²/g and 250 m²/g; a pore volume of between about 0.25 to about 0.5 cc/g; a cumulative surface area measured for all pores having a size between 20 and 40A of between 80 and 120 m²/g; and wherein said multivalent metal is present in an amount of from about 8 to about 20%.

According to another aspect of the invention, a multivalent metal-doped silicon-based gel filter medium that exhibits a breakthrough measurement for an ammonia gas/air composition of at least 60 minutes a) when present as a filter bed of 1 cm in height within a flask of a diameter of 4.1 cm, b) when exposed to a constant ammonia gas concentration of 1000 mg/m³ ammonia gas at ambient temperature and pressure, and c) when exposed simultaneously to a relative humidity of 15%; and wherein said filter medium, after breakthrough concentration of 35 mg/m³is reached, does not exhibit any ammonia gas elution in excess of said breakthrough concentration. Preferably, the breakthrough time is at least 120 minutes. Furthermore, another aspect of this invention concerns multivalent metal-doped silicon-based gel materials that exhibit a breakthrough time of at least 60 minutes when exposed to the same conditions as listed above and within the same test protocol, except that the relative humidity is 80%. Preferably, the breakthrough time for such a high relative humidity exposure test example is at least 120 minutes, as well.

Still another potential aspect of this invention is the inclusion of an oxidizing agent, such as a permanganate or peroxide, during manufacture of the gel materials. Such a component aids in capturing nitrogen dioxide and prevents conversion of that noxious gas to another noxious gas, NO, thereby increasing the viability of the overall filter medium as a decontaminant of toxic gases from certain environments.

According to still another aspect of the invention, a method of producing oxidizer- and metal-doped silicon gel-based particles is provided, said method comprising the sequential steps of:
a) providing a silicon-based gel material;
b) wet reacting said silicon-based gel material with at least one multivalent metal salt to produce metal-doped silicon-based gel material; and further reacting with at least one compound capable of acting as an oxidizer to maintain reactive species in an oxidized state;
c) drying said oxidizer- and metal-doped silicon-based gel materials. Alternatively, step “a” may include a production step for generating said silicon-based gel materials.

Additionally, it has been found that the oxidizer- and metal-doped silicon-based gel materials noted above can be pre-reduced in order to provide a more reliable filter medium in term of trapping nitrogen oxides from an airstream. In particular, the pre-reduced form of potassium permanganate, manganese dioxide, essentially, is a safer material to handle and store (in terms of stability). Prior to such a reduction step, the permanganate-treated materials will perform at a higher rate to the subsequently reduced types; however, as noted previously, during storage and handling, the potential for destabilization of the reduced forms will be drastically curtailed. Thus, another embodiment of this invention would be the provision of such a pre-reduced filter medium product.

One distinct advantage of this invention is the provision of a filter medium that exhibits highly effective ammonia uptake and breakthrough properties when present in a relatively low amount and under a pressure typical of an enclosed space and over a wide range of relative humidity. Among other advantages of this invention is the provision of a filter system for utilization within an enclosed space that exhibits a steady and effective uptake and breakthrough result for ammonia gas and that removes such noxious gases from an enclosed space at a suitable rate for reduction in human exposure below damage levels. Yet another advantage is the ability of this invention to irreversibly prevent release of noxious gases once adsorbed, under normal conditions.

Also, said invention encompasses a filter system wherein at least 15% by weight of such a filter medium has been introduced therein. Furthermore, the production of such metal-doped silica-based material gel-like particles, wherein the reaction of the metal salt is preferably performed while the gel-like particle is in a wet state has been found to be very important in provided the most efficient and thus best manner of incorporating such metal species within the micropores of the subject silica materials. As such, it was determined that such a wet gel doping step was necessary to provide the most efficient filter medium and overall filter systems for such noxious gas (such as, as one example, ammonia).

In terms of the nitrogen oxide benefits, the oxidized gel materials (both the pre-reduced and non-reduced forms) exhibit excellent removal characteristics of the highly toxic gases nitrogen dioxide and nitric oxide. The US Department of Labor Occupational Safety and Health Administration (“OSHA”) has set stringent guidelines aimed at protecting workers performing operations in an environment potentially contaminated with nitrogen oxides. The Permissible Exposure Limit (“PEL”) for NO₂ has been established at 5 ppm, 9 mg/m³ ceiling and NO at 25 ppm, 30 mg/m³. As a result, effective, low cost means of removing nitrogen oxides from ambient streams of air are needed. Of particular interest is the removal capability of nitrogen oxides simultaneously with other potentially toxic industrial chemicals like ammonia.

As noted above, impregnated, activated carbon is known to strongly adsorb a wide variety of organic chemicals from ambient air streams. Such a material is not effective at removing nitrogen oxides which are by-products of some industrial reactions. There is additionally an inherent benefit from having a combined absorption of multiple compounds from a single absorbent. Although mixtures and layered bed filters are effective, they can be complex and costly to produce. A single composite particle has distinct advantages from manufacturing, storage, and complexity perspectives, at least.

The present invention, according to one embodiment, comprises an adsorbent for removing NO₂ from air over a wide range of ambient temperatures, said process comprising contacting the air with an oxidizer impregnated high surface area silica gel alone or part of a composite matrix for a sufficient time to remove NO₂ and prevent the formation of other toxic nitrogen oxides, specifically NO.

### DETAILED DESCRIPTION OF THE INVENTION

For purposes of this invention, the term “silicon-based gel” is intended to encompass materials that are formed from the reaction of a metal silicate (such as sodium silicate) with an acid (such as sulfuric acid) and permitted to age properly to form a gel material or materials that are available from a natural source (such as from rice hulls) and exhibit pore structures that are similar to such gels as formed by the process above. Such synthetic materials may be categorized as either silicic acid or polysilicic acid types or silica gel types, whereas the natural source materials are typically harvested in a certain form and treated to ultimately form the final gel-like product (such a method is provided within U.S. Pat. No. 6,638,354). The difference between the two synthetic categories lies strictly within the measured resultant pH level of the gel after reaction, formation and aging. If the gel exhibits a pH of below 3.0 after that stage, the gel is considered silicic or polysilicic acid in type. If pH 3.0 or above, then the material is considered a (traditional) silica gel. In any event, as noted above, the term “silicon-based gel” is intended to encompass both of these types of gel materials. It has been found that silicon-based gels exhibiting a resultant pH of less than 3.0 (silicic or polysilicic acid gels) contain a larger percentage of micropores of size less than 20′ with a median pore size of about 30′, while silicon-based gels exhibiting a higher acidic pH, such as pH of 3.0 and above (preferably, though not necessarily, as high as 4) contain a mixture of pore sizes having a median pore size of about 30′ to about 60′. While not wishing to be held by theory, it is believed that capture of toxic gases, such as ammonia, is accomplished by two separate (but potentially simultaneous) occurrences within the pores of the metal-doped silicon-based gels: acid-base reaction and complexation reaction. Thus silicon-based gels formed at pH<2 contain more residual acid than the gels formed at pH 3-4, however the gels formed at pH 3-4 contain more pores of size suitable to entrap a metal, such as copper, and thus have more metal available for a complexation reaction. It is believed that the amount of a gas such as ammonia that is captured and held by the silicon-based gel results from a combination of these two means. The term “multivalent metal salt” is intended to include any metal salt having a metal exhibiting a valence number of at least three. Such a multivalent metal is particularly useful to form the necessary complexes with ammonia; a valence number less than three will not readily form such complexes.

The hydrous silicon-based gels that are used as the base materials for metal doping as well as the basic materials for the desired air filtration medium may be prepared from acid-set silica hydrogels. Silica hydrogel may be produced by reacting an alkali metal silicate and a mineral acid in an aqueous medium to form a silica hydrosol and allowing the hydrosol to set to a hydrogel. When the quantity of acid reacted with the silicate is such that the final pH of the reaction mixture is acidic, the resulting product is considered an acid-set hydrogel. Sulfuric acid is the most commonly used acid, although other mineral acids such as hydrochloric acid, nitric acid, or phosphoric acid may be used. Sodium or potassium silicate may be used, for example, as the alkali metal silicate. Sodium silicate is preferred because it is the least expensive and most readily available. The concentration of the aqueous acidic solution is generally from about 5 to about 70 percent by weight and the aqueous silicate solution commonly has an SiO₂ content of about 6 to about 25 weight percent and a molar ratio of SiO₂ to Na₂O of from about 1:1 to about 3.4:1.

The alkali metal silicate solution is added to the mineral acid solution to form a silica hydrosol. The relative proportions and concentrations of the reactants are controlled so that the hydrosol contains about 6 to about 20 weight percent SiO₂ and has a pH of less than about 5 and commonly between about 1 to about 4. Generally, continuous processing is employed and alkali silicate is metered separately into a high speed mixer. The reaction may be carried out at any convenient temperature, for example, from about 15 to about 80° C. and is generally carried out at ambient temperatures.

The silica hydrosol will set to a hydrogel in generally about 5 to about 90 minutes and is then washed with water or an aqueous acidic solution to remove residual alkali metal salts which are formed in the reaction. For example, when sulfuric acid and sodium silicate are used as the reactants, sodium sulfate is entrapped in the hydrogel. Prior to washing, the gel is normally cut or broken into pieces in a particle size range of from about ½ to about 3 inches. The gel may be washed with an aqueous solution of mineral acid such as sulfuric acid, hydrochloric acid, nitric acid, or phosphoric acid or a medium strength acid such as formic acid, acetic acid, or propionic acid.

Generally, the temperature of the wash medium is from about 27° C. to about 93° C. Preferably, the wash medium is at a temperature of from about 27° C. to about 38° C. The gel is washed for a period sufficient to reduce the total salts content to less than about 5 weight percent. The gel may have, for example, a Na₂O content of from about 0.05 to about 3 weight percent and a SO₄ content of from about 0.05 to about 3 weight percent, based on the dry weight of the gel. The period of time necessary to achieve this salt removal varies with the flow rate of the wash medium and the configuration of the washing apparatus. Generally, the period of time necessary to achieve the desired salt removal is from about 0.5 to about 3 hours. Thus, it is preferred that the hydrogel be washed with water at a temperature of from about 27° C. to about 38° C. for about 0.5 to about 3 hours. In one potential embodiment, the washing may be limited in order to permit a certain amount of salt (such as sodium sulfate), to be present on the surface and within the pores of the gel material. Such salt is believed, without intending on being limited to any specific scientific theory, to contribute a level of hydration that may be utilized for the subsequent metal doping procedure to effectively occur as well as contributing sufficient water to facilitate complexation between the ammonia gas and the metal itself upon exposure.

In order to prepare hydrous silicon-based gels suitable for use in the filter media of this invention, the final gel pH upon completion of washing as measured in 5 weight percent aqueous slurry of the gel, may range from about 1.5 to about 5.

The washed silica hydrogel generally has a water content, as measured by oven drying at 105° C. for about 16 hours, of from 10 to about 60 weight percent and a particle size ranging from about 1 micron to about 50 millimeters. Alternatively the hydrogel is then dewatered to a desired water content of from about 20 to about 90 weight percent, preferably from about 50 to about 85 weight percent. Any known dewatering method may be employed to reduce the amount of water therein or conversely increase the solids content thereof. For example, the washed hydrogel may be dewatered in a filter, rotary dryer, spray dryer, tunnel dryer, flash dryer, nozzle dryer, fluid bed dryer, cascade dryer, and the like.

The average particle size referred to throughout this specification is determined in a MICROTRAC® particle size analyzer. When the water content of the hydrogel is greater than about 90 weight percent, the hydrogel may be pre-dried in any suitable dryer at a temperature and for a time sufficient to reduce the water content of the hydrogel to below about 85 weight percent to facilitate handling, processing, and subsequent metal doping.

Generally, the hydrogel materials after formation and aging are of very coarse sizes and thus should be broken apart to facilitate proper metal impregnation. Such a size reduction may be accomplished by various methods, including milling, grinding, and the like. One option, however, is to subject the hydrogel materials to high shear mixing during the metal doping procedure. In such a step, the particle sizes can be reduced to the sizes necessary for proper filter utilization. Alternatively, the hydrogel particles may be ground to relatively uniform particles sizes concurrently during doping or subsequent to the doping step. In such alternative manners, the overall production method can effectuate the desired homogeneous impregnation of the metal for the most effective noxious gas removal upon utilization as a filter medium.

Thus, in one possible embodiment, the silica hydrogel is wet ground in a mill in order to provide the desired average particle size suitable for further reaction with the metal dopant and the subsequent production of sufficiently small pore sizes for the most effective ammonia gas trapping and holding while present within a filter medium. For example, the hydrogels may be concurrently ground and dried with any standard mechanical grinding device, such as a hammer mill, as one non-limiting example. The ultimate particle sizes of the multivalent-metal impregnated (doped) silicon-based gel materials are dependent upon the desired manner of providing the filter medium made therefrom. Thus, packed media will require larger particle sizes (from 10 to 100 microns, for example) whereas relatively small particles sizes (from 1 to 20 microns, for example) may be utilized as extrudates within films or fibers. The important issue, however, is not the particle sizes in general, but the degree of homogeneous metal doping effectuated within the pores of the subject hydrogels themselves.

The hydrous silicon-based gel product after grinding preferably remains in a wet state (although drying and grinding may be undertaken, either separately or simultaneously; preferably, though, the materials remain in a high water-content state for further reaction with the metal dopant) for subsequent doping with metal salts or oxidizers in order to provide effective toxic chemical trapping and holding capability within a filter medium. Such a wet state reaction is thus encompassed within the term “wet reaction” or “wet react” for this invention. Without intending on being bound to any specific scientific theory, it is believed that the wet state doping permits incorporation of sufficient chemical species within the pores of the silicon-based gel product to permit sufficient points for reaction, complexation or entrapment of the target toxic chemicals. In a wet state, the pores of the subject silicon-based gel product are large enough in volume to allow for a metal salt or chemical moiety to enter therein. Subsequent drying thus appears to shrink the pores around the resultant compound to a volume that, upon introduction of target toxic gas, causes the gas to condense into a liquid. It is apparently this liquid that then exists within the small volume pores that will contact with the chemical species to effectuate said removal. Thus, it is believed that the production of small volume pores around the chemical species therein to a level wherein the remaining volume within such pores is small enough to permit such condensation of the target toxic chemical species followed by reliable contact for the needed substantially permanent removal for effective capture of the molecules is best provided through the wet state reaction noted above. Included as one possible alternative within the term “wet reaction” or “wet react” is the ability to utilize gel particles that have been dried to a certain extent and reacted with an aqueous solution of chemical impregnants in a slurry. Although the resultant performance of such an alternative filter medium does not equal that of the aforementioned product of pre-dried, wet, gel particles with a metal salt, such a filter medium does exhibit performance results that exceed gels alone, or dry-mixed metal-treated salt materials. Such an alternative method has proven effective and is essential when utilizing the natural source materials (from rice hulls, for example, and as noted above) as reactants with an aqueous impregnant solution.

The metals that can be utilized for such a purpose include, as alluded to above, any multivalent metal, such as, without limitation, cobalt, iron, manganese, zinc, aluminum, chromium, copper, tin, antimony, indium, tungsten, silver, gold, platinum, mercury, palladium, cadmium, and nickel. For cost reasons, copper and zinc are potentially preferred, with copper most preferred. The listing above indicates the metals possible for production during the doping step within the pores of the subject silicon-based gel materials. The metal salt is preferably water-soluble in nature and facilitates dissociation of the metal from the anion when reacted with silica-based materials. Thus, sulfates, chlorides, bromides, iodides, nitrates, and the like, are possible as anions, with sulfate, and thus copper sulfate, most preferred as the metal doping salt (cupric chloride is also potentially preferred as a specific compound; however, the acidic nature of such a compound may militate against use on industrial levels). Without intending on being bound to any specific scientific theory, it is believed that copper sulfate enables doping of copper [as a copper (II) species] in some form to the silicon-based gel structure, while the transferred copper species maintains its ability to complex with ammonium ions, and further permits color change within the filter medium upon exposure to sufficient amounts of ammonia gas to facilitate identification of effectiveness of gas removal and eventual saturation of the filter medium. In such a manner, it is an easy task to view the resultant filtration system empirically to determine if and when the filter medium has been saturated and thus requires replacement.

The wet state doping procedure has proven to be particularly useful for the provision of certain desired filter efficiency results, as noted above. A dry mixing of the metal salt and silicon-based gel does not accord the same degree of impregnation within the gel pores necessary for ammonia capture and retention. Without such a wet reaction, although capture may be accomplished, the ability to retain the trapped ammonia (in this situation, the ammonia may actually be modified upon capture or within the subject environment to ammonium hydroxide as well as a portion remain as ammonia gas) can be reduced. It is believed, without intending on being limited to such a theory, that in such a product, ammonia capture is still effectuated by metal complexation, but the lack of small pore volumes with metal incorporated therein limits the ability for the metal to complex strongly enough to prevent release upon certain environmental changes (such as, as one example, high temperature exposure). Such a result is actually the object of the closest prior art. As in the noted Kemira reference above, a dry mix procedure produces a regenerable filter medium rather than a permanent capture and retention filter medium. The particular wet reaction is discussed more specifically within the examples below, but, in its broadest sense, the reaction entails the reaction of a silicon-based gel with introduced water present in an amount of at least 50% by weight of the gel and metal salt materials. Preferably, the amount of water is higher, such as at least 70%; more preferably at least 80%, and most preferably at least 85%. If the reaction is too dry, proper metal doping will not occur as the added water is necessary to transport the metal salts into the pores of the gel materials. Without sufficient amounts of metal within such pores, the gas removal capabilities of the filter medium made therefrom will be reduced. The term “added” or “introduced” water is intended to include various forms of water, such as, without limitation, water present within a solution of the metal salt or the gel, hydrated forms of metal salts, hydrated forms of residual gel reactant salts, such as sodium sulfate, moisture, and relative humidity; basically any form that is not present as an integral part of the either the gel or metal salt itself, or that is not transferred into the pores of the material after doping has occurred. Thus, as non-limiting examples, again, the production of gel material, followed by drying initially with a subsequent wetting step (for instance, slurrying within an aqueous solution, as one non-limiting example), followed by the reaction with the multivalent metal salt, may be employed for this purpose, as well as the potentially preferred method of retaining the gel material in a wet state with subsequent multivalent metal salt reaction thereafter.

Water is also important, however, to aid in the complexation of the metal with the subject noxious gas within the gel pores. It is believed, without intending on being bound to any specific scientific theory, that upon doping the metal salt is actually retained but complexed, via the metal cation, to the silicon-based gel within the pores thereof (and some may actual complex on the gel surface but will more readily become de-complexed and thus removed over time; within the pores, the complex with the metal is relatively strong and thus difficult to break). The presence of water at that point aids in removing the anionic portion of the complexed salt molecule through displacement thereof with hydrates. It is believed that these hydrates can then be displaced themselves by, as one example, the ammonia gas (or ammonium ions) thereby producing an overall gel/metal/ammonium complex that is strongly associated and very difficult to break, ultimately providing not only an effective ammonia gas capture mechanism, but also a manner of strongly retaining such ammonia gases. The water utilized as such a complexation aid can be residual water from the metal doping step above, or present as a hydrated form on either the gel surface (or within the gel pores) or from the metal salt reactant itself. Furthermore, and in one potentially preferred embodiment, such water may be provided through the presence of humectants (such as glycerol, as one non-limiting example).

Furthermore, of importance as well is the potentially preferred embodiment of contacting and/or reacting the gel material with an oxidizing agent to provide extra nitrogen oxide removal capabilities. Any oxidizing material within those categorized in Classes 1 through 4 would be suitable, with Class 1 and 2 types preferred due to safety issues in handling during incorporation. Examples of Class 1 types include aluminum nitrate, potassium dichromate, ammonium persulfate, potassium nitrate, barium chlorate, potassium persulfate, barium nitrate, silver nitrate, barium peroxide, sodium carbonate peroxide, calcium chlorate, sodium dichloro-s-triazinetrione, calcium nitrate, sodium dichromate, calcium peroxide, sodium nitrate, cupric nitrate, sodium nitrite, hydrogen peroxide (8-27.5%), sodium perborate, lead nitrate, sodium perborate tetrahydrate, lithium hypochlorite, sodium perchlorate monohydrate, lithium peroxide, sodium persulfate, magnesium nitrate, strontium chlorate, magnesium perchlorate, strontium nitrate, magnesium peroxide, strontium peroxide, nickel nitrate, zinc chlorate, nitric acid (<70% conc.), zinc peroxide, and perchloric acid (<60% concen.). Examples of Class 2 types include calcium hypochlorite (<50% wgt), potassium permanganate, chromium trioxide (chromic acid), sodium chlorite (<40% wgt.), halane, sodium peroxide, hydrogen peroxide (27.5-52% conc.), sodium permanganate, nitric acid (>70% conc.), and trichloro-s-triazinetrione. Examples of Class 3 types include ammonium dichromate, potassium chlorate, hydrogen peroxide (52-91% conc.), potassium dichloroisocyanurate, calcium hypochlorite (>50% wgt.), sodium chlorate, perchloric acid (60-72.5% conc.), sodium chlorite (>40% wgt.), potassium bromate, and sodium dichloro-s-triazinetrione. Examples of Class 4 types include ammonium perchlorate, ammonium permanganate, guanidine nitrate, hydrogen peroxide (>91% cone.), perchloric acid (>72.5%), and potassium superoxide. Preferably the oxidizing material is potassium permanganate or calcium peroxide. The amount of oxidizing agent contacted there with the gel material particles is from 0.1 to 10%. The contacting/reacting may occur during gel production or, and preferably, thereafter, in order to allow sufficient amount of oxidizing agent to attach to sites on the gel surfaces.

As noted above, due to the potential instability of certain oxidizer compounds (most notably potassium permanganate), it has also been determined that subsequent to oxidizer doping of the target silicon-based gel materials, the resultant composites can be reduced (such as at high temperatures) to effectuate reduction of the oxidizer dopant to a pre-reduced form. It is believed, without intending on relying upon any specific scientific basis, that a permanganate dopant will become reduced at a manganese dioxide material while present on the surface of the target gel materials, as one example. Upon exposure to a contaminated airstream, the pre-reduced component will still act as an oxidizer, thereby providing some level of nitrogen oxide removal characteristics as discussed above, albeit less than the permanganate form.

The inventive silicon-based gel particles thus have been doped (impregnated) with at least one multivalent metal salt (such as, as one non-limiting example, copper sulfate) in an amount of from about 2 to about 30 wt %, expressed as the percentage weight of base metals, such as copper, of the entire dry weight of the metal-impregnated (doped) silicon gel-based particles. Such resultant metal-doped silicon-based gel materials thus provide a filter medium that exhibits a prolonged breakthrough time to the exposure limit of 35 mg/m³ for an ammonia gas/air composition having a 1000 mg/m³ ammonia gas concentration when exposed to ambient pressure (i.e., from 0.8 to 1.2 atmospheres, or roughly from 0.81 to 1.25 kPa) and temperature (i.e., from 20-25° C.) when applied to a filter bed of at most 2 cm height within a cylindrical tube of 4.1 cm in diameter, and wherein said ammonia gas captured by said filter medium does not exhibit any appreciable regeneration upon exposure to ambient temperature and pressure for 72 hours. And, alternatively, the gel materials also have the aforementioned oxidizer thereon for removal of nitrogen oxides from an environment. Such resultant oxidizer metal-doped silicon-based gel materials thus provide a filter medium that exhibits a prolonged breakthrough time to the exposure limit of 35 mg/m³ for an ammonia gas/air composition having a 1000 mg/m³ ammonia gas concentration when exposed to ambient pressure (i.e., from 0.8 to 1.2 atmospheres, or roughly from 0.81 to 1.25 kPa) and temperature (i.e., from 20-25° C.) when applied to a filter bed of at most 2 cm height within a cylindrical tube of 4.1 cm in diameter, and wherein said ammonia gas captured by said filter medium does not exhibit any appreciable regeneration upon exposure to ambient temperature and pressure for 72 hours. And exhibits a prolonged breakthrough time to the exposure limit of 9 mg/m³ NO₂ for an nitrogen oxides/air composition having a 375 mg/m³ NO₂ gas concentration when exposed to ambient pressure (i.e., from 0.8 to 1.2 atmospheres, or roughly from 0.81 to 1.25 kPa) and temperature (i.e., from 20-25° C.) when applied to a filter bed of at most 2 cm height within a flask of 4.1 cm in diameter, and wherein said NO₂ gas captured by said filter medium does not exhibit any appreciable regeneration upon exposure to ambient temperature and pressure for 72 hours. This absorbent also exhibits a prolonged breakthrough time to the exposure limit of 30 mg/m³ for an nitric oxide (NO) that may be present as a contaminant or result from an uncontrolled reaction when exposed to ambient pressure (i.e., from 0.8 to 1.2 atmospheres, or roughly from 0.81 to 1.25 kPa) and temperature (i.e., from 20-25° C.) when applied to a filter bed of at most 2 cm height within a flask of 4.1 cm in diameter, and wherein said NO₂ and NO gases captured by said filter medium does not exhibit any appreciable regeneration upon exposure to ambient temperature and pressure for 72 hours.

The hydrous silicon-based gels (and oxidizer and pre-reduced oxidizer metal-treated gels as well) are employed in the filter medium of this invention in an amount from about 1 to about 90 percent, preferably about 5 to about 70 percent, by weight of the entire filter medium composition.

The filter medium of the invention can also further contain as optional ingredients, silicates, clays, talcs, aluminas, carbons, polymers, including but not limited to polysaccharides, gums or other substances used as binder fillers. These are conventional components of filter media, and materials suitable for this purpose need not be enumerated for they are well known to those skilled in the art. Furthermore, such metal-doped silicon-based gels of the invention may also be introduced within a polymer composition (through impregnation, or through extrusion) to provide a polymeric film, composite, or other type of polymeric solid for utilization as a filter medium. Additionally, a nonwoven fabric may be impregnated, coated, or otherwise treated with such invention materials, or individual yarns or filaments may be extruded with such materials and formed into a nonwoven, woven, or knit web, all to provide a filter medium base as well. Additionally, the inventive filter media may be layered within a filter canister with other types of filter media present therewith (such as layers of carbon black material), or, alternatively, the filter media may be interspersed together within the same canister. Such films and/or fabrics, as noted above, may include discrete areas of filter medium, or the same type of interspersed materials (carbon black mixed on the surface, or co-extruded, as merely examples, within the same fabric or film) as well.

The filter system utilized for testing of the viability of the medium typically contains a media bed thickness of from about 1 cm to about 3 cm thickness, preferably about 1 cm to about 2 cm thickness within a cylindrical tube of 4.1 cm in diameter. Without limitation, typical filters that may actually include such a filter medium, for example, for industrial and/or personal use, will comprise greater thicknesses (and thus amounts) of such a filter medium, from about 1-15 cm in thickness and approximately 10 cm in diameter, for example for personal canister filter types, up to 100 cm in thickness and 50 cm in diameter, at least, for industrial uses. Again, these are only intended to be rough approximations for such end use applications; any thickness, diameter, width, height, etc., of the bed and/or the container may be utilized in actuality, depending on the length of time the filter may be in use and the potential for gaseous contamination the target environment may exhibit. The amount of filter medium that may be introduced within a filter system in any amount, as long as the container is structurally sufficient to hold the filter medium therein and permits proper airflow in order for the filter medium to properly contact the target gases.

It is important to note that although ammonia (and, in some instances, nitrogen oxide) gases are the test subject for removal by the inventive filter media discussed herein, such media may also be effective in removing other noxious gases from certain environments as well, including formaldehyde and amines as merely examples.

As previously mentioned, the filter medium can be used in filtration applications in an industrial setting (such as protecting entire industrial buildings or individual workers, via masks), a military setting (such as filters for vehicles or buildings or masks for individual troops), commercial/public settings (office buildings, shopping centers, museums, governmental locations and installations, and the like). Specific examples may include, without limitation, the protection of workers in agricultural environments, such as within poultry houses, as one example, where vast quantities of ammonia gas can be generated by animal waste. Thus, large-scale filters may be utilized in such locations, or individuals may utilize personal filter apparatuses for such purposes. Furthermore, such filters may be utilized at or around transformers that may generate certain noxious gases. Generally, such inventive filter media may be included in any type of filter system that is necessary and useful for the removal of potential noxious gases in any type of environment.

### Preferred Embodiments of the Invention

The % solids of the adsorbent wet cake were determined by placing a representative 2 g sample on the pan of a CEM 910700 microwave balance and drying the sample to constant weight. The weight difference is used to calculate the % solids content. Pack or tapped density is determined by weighing 100.0 grams of product into a 250-mL plastic graduated cylinder with a flat bottom. The cylinder is closed with a rubber stopper, placed on the tap density machine and run for 15 minutes. The tap density machine is a conventional motor-gear reducer drive operating a cam at 60 rpm. The cam is cut or designed to raise and drop the cylinder a distance of 2.25 in. (5.715 cm) every second. The cylinder is held in position by guide brackets. The volume occupied by the product after tapping was recorded and pack density was calculated and expressed in g/ml.

The conductivity of the filtrate was determined utilizing an Orion Model 140 Conductivity Meter with temperature compensator by immersing the electrode epoxy conductivity cell (014010) in the recovered filtrate or filtrate stream. Measurements are typically made at a temperature of 15-20° C.

### Ammonia Breakthrough

The general protocol utilized for breakthrough measurements involved the use of two parallel flow systems having two distinct valves leading to two distinct adsorbent beds (including the filter medium), connected to two different infrared detectors followed by two mass flow controllers. The overall system basically permitting mixing of ammonia and air within the same pipeline for transfer to either adsorbent bed or continuing through to the same gas chromatograph. In such a manner, the uptake of the filter media within the two adsorbent beds was compared for ammonia concentration after a certain period of time through the analysis via the gas chromatograph as compared with the non-filtered ammonia/air mixture produced simultaneously. A vacuum was utilized at the end of the system to force the ammonia/air mixture through the two parallel flow systems as well as the non-filtered pipeline with the flow controlled using 0-50 SLPM mass flow controllers.

To generate the ammonia/air mixture, two mass flow controllers generated challenge concentration of ammonia, one being a challenge air mass flow controller having a 0-100 SLPM range and the other being an ammonia mass flow controller having a 0-100 sccm range. A third air flow controller was used to control the flow through a heated water sparger to control the challenge air relative humidity (RH). Two dew point analyzers, one located in the challenge air line above the beds and the other measuring the effluent RH coming out of one of the two filter beds, were utilized to determine the RH thereof (modified for different levels).

The beds were 4.1 cm glass tubes with a baffled screen to hold the adsorbent. The adsorbent was introduced into the glass tube using a fill tower to obtain the best and most uniform packing each time.

The challenge chemical concentration was then measured using an HP 5890 gas chromatograph with a Thermal Conductivity Detector (TCD). The effluent concentration of ammonia was measured using an infrared analyzer (MIRAN), previously calibrated at a specific wavelength for ammonia.

The adsorbent was prepared for testing by screening all of the particles below 40 mesh (−425 microns). The largest particles were typically no larger than about 20 mesh (−850 microns). The valves above the two beds were initially closed. The diluent air flow and the water sparger air flow were started and the system was allowed to equilibrate at the desired temperature and relative humidity (RH). The valves above the beds were then changed and simultaneously the chemical flow was started and kept at a rate of 5.22 SLPM. The chemical flow was set to achieve the desired challenge chemical concentration. The feed chemical concentration was constantly monitored using the GC. The effluent concentrations from the two adsorbent beds (filter media) were measured continuously using the previously calibrated infrared detectors. The breakthrough time was defined as the time when the effluent chemical concentration equaled the target breakthrough concentration. For ammonia tests, the challenge concentration was 1,000 mg/m³ at 25° C. and the breakthrough concentration was 35 mg/m³ at 25° C. Ammonia breakthrough was then measured for distinct filter medium samples, with the bed depth of such samples modified as noted, the relative humidity adjusted, and the flow units of the ammonia gas changed to determine the effectiveness of the filter medium under different conditions. A breakthrough time in excess of 40 minutes was targeted.

In a similar manner, using methods described above, the breakthrough time for nitrogen oxides were determined. The chemical flow was set to achieve the desired challenge chemical concentration by diluting NO₂ gas to a concentration of 375 mg/m3 with air at the specified relative humidity level. The feed chemical concentration was monitored using a chemiluminescence detector. The effluent concentration from the adsorbent bed (filter media) was measured continuously using the previously calibrated chemiluminescence detector to measure simultaneously, NO₂, NO and NOx. The breakthrough time was defined as the time when the effluent chemical concentration equaled the target breakthrough concentration. For NOx tests, the challenge concentration was 375 mg/m³ at 25° C. and the breakthrough concentration was 30 mg/m³ at 25° C. for NO and 9 mg/m³ at 25° C. for NO₂.

The breakthrough requirements are summarized in Table 1, below.

TABLE 1
Ammonia and Nitrogen Oxides Breakthrough Targets

| | Breakthrough Concentration, | Target Breakthrough time, |
|---|---|---|
| | mg/m3 | minutes |
| NH₃ | 35 | 40 |
| NO₂ | 9 | 15 |
| NO | 30 | 15 |

### Nitrogen Oxide Removal—Metal Oxidizer-Treated Gel Production

The preferred embodiments including an oxidizing material and/or a pre-reduced oxidizing material for nitrogen oxide removal are provided as follows:

### INVENTIVE EXAMPLE 1

Particles of silicon-based gel were produced by adding a solution of 11.4% sulfuric acid solution to 2000 mL 24.7% sodium silicate (3.3 mole ratio) solution with agitation at 300-400 rpm until the pH of the solution reached the target pH of 3.0. The suspension was then discharged into 5000 ml deionized water at 85° C. for the 30 minutes to complete gel formation. The gel cake was recovered by filtration to form a mass of gel particles with conductivity of less than 3000 μS. Next, the gel was broken apart with further agitation. The washed particles are then filtered and collected and the resulting particles were dried in an oven set at 105° C. for 16 hours. To form granules and increase product density, 200 g of the dried blend prepared above were compacted in a roller compactor (TF-Labo available from Vector Corporation) using a pressing force 7 bar to form crayon-shaped agglomerates, which were then sized by sieving to recover granules sized between 850 μm and 425 μm.

### INVENTIVE EXAMPLE 2

Wet gel cake from Example 1 was impregnated with copper by adding 1500 g amount of gel wet cake formed above (17.35% solids) and 500g of deionized water. To this add 1.3 g 98% H2SO4 and 390 g of CuSO₄.5H₂O. (The % solids of the dried gel, determined according to the method described above, was used to estimate the quantity of impregnate required to achieve the desired metal level.) The slurry was then agitated at 3000 rpm for 15 minutes at ambient temperature. The uniform slurry was then placed directly in an oven set at 105° C. and dried overnight (16 hours). To form granules and increase product density, 200 g of the dried blend prepared above were compacted in a roller compactor (TF-Labo available from Vector Corporation) using a pressing force 7 bar to form crayon-shaped agglomerates, which were then sized by sieving to recover granules sized between 850 μM and 425 μm.

### INVENTIVE EXAMPLE 3

To 612 g of silicic acid gel from Example 1 having a solids concentration of 16.35%, add 4 g of KMnO₄ crystals. Blend with a high shear mixer to form a homogeneous slurry. Recover and dry for 16 h at 105° C. To form hard granules and increase product density, 100 g of the dried blend prepared above were compacted in a roller compactor (TF-Labo available from Vector Corporation) using a pressing force 7 bar to form crayon-shaped agglomerates, which were then sized by sieving to recover granules sized between 850 μm and 425 μm.

### INVENTIVE EXAMPLE 4

To 100 g of dried silicic acid gel from Example 1, add 4 g calcium peroxide powder and 10 g deionized water dropwise while dispersing in Cuisinart® blender to effect a homogeneous powder. To form hard granules and increase product density, 100 g of the dried blend prepared above were compacted in a roller compactor (TF-Labo available from Vector Corporation) using a pressing force 7 bar to form crayon-shaped agglomerates, which were then sized by sieving to recover granules sized between 850 μm and 425 μm.

### INVENTIVE EXAMPLE 5

The copper impregnated gel of Example 2 was doped with potassium permanganate by mixing 455 g of Example 2 slurry (22.45% solids) with 4 g KMnO₄ crystals. The slurry was stirred at 2000 rpm for 20 minutes and dried in an oven for 16 hours at 100° C. To form hard granules and increase product density, 100 g of the dried blend prepared above were compacted in a roller compactor (TF-Labo available from Vector Corporation) using a pressing force 7 bar to form crayon-shaped agglomerates, which were then sized by sieving to recover granules sized between 850 μm and 425 μm.

### INVENTIVE EXAMPLE 6

The copper impregnated gel of Example 2 was doped with potassium permanganate by mixing 910 g of Example 2 slurry (22.45% solids) with 8 g KMnO₄ crystals. Using methods described in Example 2, the slurry was dried at 90° C. and sized granules were produced.

### INVENTIVE EXAMPLE 7

The wet gel cake from Example 3 was impregnated with KMnO₄ by adding 4 g of KMnO₄ crystals to 612 g of the gel wet cake containing 16.35% solids. This mixture was blended using a high shear mixer to form a homogeneous slurry, which was recovered and dried for 16 h at 105° C. To form granules and increase product density, the dried blend prepared above was compacted in a roller compactor (TF-Labo available from Vector Corporation) using a pressing force 7 bar to form crayon-shaped agglomerates, which were then sized by sieving to recover 20×40 US standard mesh size (between 850 μm and 425 μm) granules.

### INVENTIVE EXAMPLE 8

The dry silicic acid gel from Example 3 was impregnated with calcium peroxide by adding a solution containing 4 g of calcium peroxide and 10 g of deionized water dropwise to 100 g of the dried silicic acid gel under shear using a Cuisinart® food processor to create a homogeneous powder. To form granules and increase product density, the dried blend prepared above was compacted in a roller compactor (TF-Labo available from Vector Corporation) using a pressing force 7 bar to form crayon-shaped agglomerates, which were then sized by sieving to recover 20×40 US standard mesh size (between 850 μm and 425 μm) granules.

### INVENTIVE EXAMPLE 9

The copper impregnated gel from Example 4 was doped with KMnO₄ by mixing 455 g of Example 4 slurry containing 22.45% solids with 4 g KMnO₄ crystals. The slurry was stirred at 2000 rpm for 20 minutes and dried in an oven for 16 hours at 100° C. To form granules and increase product density, the dried blend prepared above was compacted in a roller compactor (TF-Labo available from Vector Corporation) using a pressing force 7 bar to form crayon-shaped agglomerates, which were then sized by sieving to recover 20×40 US standard mesh size (between 850 μm and 425 μm) granules.

### INVENTIVE EXAMPLE 10

6.768 lbs of (30:1/SiO₂:Al₂O₃) H-ZSM-5 zeolite from Zeolyst Incorporated (Zeolyst 3020E) was mixed on low speeds with 0.384 lbs of bentonite in an Eirich high shear mixer. To the dry ingredients was then added 0.576 lbs of 68-70% HNO₃ diluted in 1.8 lbs of deionized water. After all the acidified water was added, another 1.8 lbs of deionized water was added to the spinning mixture. The mixture was spun on high rotor and bowl speed until fine granules were formed (for approximately 40 minutes). The mixture was then dried in an oven at 85 to 150° C. until a final moisture of ˜10% was reached. The dried granules were then sized by sieving to recover 25×40 US standard mesh size (between 700 μm and 425 μm) granules.

### INVENTIVE EXAMPLE 11

506 lbs of 15% copper silica gel slurry and 4.59 lbs of KMnO₄ were mixed to produce a slurry. This slurry of potassium permanganate copper silica gel was then fed to a Pulvocron 20 machine via a pump. The gel slurry was then dried to both remove moisture and to reduce the permanganate component. The slurry was fed through the Pulvocron 20 machine at a rate of 200 lb/hr dry product with 317 lbs/hr of slurry @˜21% solisa at a inlet temperature of 676° F. and discharge air temperature of 150° F. The resultant materials had a moisture content of 14.4% and were colored brown, thereby indicating a chemical change in the previous potassium permanganate surface treatment to a reduced state, most likely manganese dioxide. The dried blend prepared above was granulated on a Bepex Model MS-75 compactor. The material was fed using a high compression screw feed at feed rates of 2.8 rpm roll speed and 29 rpm feed screw speed with a roll pressure setting of 2,500 psi. The flake produced from the rolls was 3″ long ribbons and splinters. The compression process heated up the material significantly, likely with some moisture loss. A 5/64″ disintegrator screen was used to cut the material down to target size, after which it was screened on a 60″ Sweco vibratory screen to recover 20×40 US standard mesh size (between 850 μm and 425 μm) granules.

### COMPARATIVE EXAMPLE 1

20×40 US standard mesh size (between 850 μm and 425 μm) particles of commercially available ASZM-TEDA carbon from Calgon Incorporated, were procured.

### COMPARATIVE EXAMPLE 2

Particles of commercially available (30:1/SiO₂:Al₂O₃) H-ZSM-5 zeolite from Zeolyst Incorporated, were procured. To form granules and increase product density, these particles were compacted in a roller compactor (TF-Labo available from Vector Corporation) using a pressing force 7 bar to form crayon-shaped agglomerates, which were then sized by sieving to recover 20×40 US standard mesh size (between 850 μm and 425 μm) granules.

### COMPARATIVE EXAMPLE 3

Particles of silicon-based gel were produced by adding a solution of 11.4% sulfuric acid solution to 2000 ml 24.7% sodium silicate (3.3 mole ratio) solution with agitation at 300-400 rpm until the pH of the solution reached the target pH of 3.0. The suspension was then discharged into 5000 ml deionized water at 85° C. for the 30 minutes to complete gel formation. The gel cake was recovered by filtration to form a mass of gel particles with conductivity of less than 3000 μS. Next, the gel was broken apart with further agitation. The washed particles are then filtered and collected and the resulting particles were dried in an oven set at 105° C. for 16 hours.

### COMPARATIVE EXAMPLE 4

The wet gel cake from Example 3 was impregnated with copper by mixing 1500 g of the gel wet cake containing 17.35% solids and 500 g of deionized water, to which 1.3 g of 98% H₂SO₄ and 390 g of CuSO₄.5H₂O was added. (The % solids of the dried gel was used to estimate the quantity of impregnate required to achieve the desired metal level). The slurry was then agitated at 3000 rpm for 15 minutes at ambient temperature. The homogeneous slurry was then placed directly in an oven set at 105° C. and dried overnight for 16 hours. To form granules and increase product density, the dried blend prepared above was compacted in a roller compactor (TF-Labo available from Vector Corporation) using a pressing force 7 bar to form crayon-shaped agglomerates, which were then sized by sieving to recover 20×40 US standard mesh size (between 850 μm and 425 μm) granules.

### Comparative Example 5

To 94.00 g of (30: 1/SiO₂:Al₂O₃) H-ZSM-5 zeolite from Zeolyst Incorporated (Zeolyst 3020E), 8.57 g of 68-70% HNO₃ diluted in 31.98 g of de-ionized water was added under shear in a Cuisinart® food processor to form granules. After the granules formed they were then sized by sieving to recover 20×40 US standard mesh size (between 850 μm and 425 μm) granules, which were then dried in an oven at 85 to 105° C. until a final moisture of 10±2% was reached.

### COMPARATIVE EXAMPLE 6

To 97.00 g of (30 :1/SiO₂:Al₂O₃) H-ZSM-5 zeolite from Zeolyst Incorporated (Zeolyst 3020E), 4.29 g of 68-70% HNO₃ diluted in 31.51 g of de-ionized water was added under shear in a Cuisinart® food processor to form granules. After the granules formed they were then sized by sieving to recover 20×40 US standard mesh size (between 850 μm and 425 μm) granules, which were then dried in an oven at 85 to 105° C. until a final moisture of 10±2% was reached.

### NO Breakthrough Testing

To test for such breakthrough measurements, cylindrical filters (4.08 cm diameter) with either single or stacked filter medium configurations were provided. Each bed medium was 1 cm in depth. If stacked, two or more beds were present each at 1 cm apiece, with a screen in place placed thereafter in the direction of the feed gas. Each sample tested is delineated in the following testing examples, with constant values for NO₂ feed concentration (200 ppm), relative humidity (15±2%), feed gas flow rate (at the top of the bed)(5.22 SLPM), and resultant media velocity (−6.6 cm/sec) in each instance. The measured results are provided in Table 2, below.

### NO REMOVAL EXAMPLE A

NOₓ test of a moisture unequilibrated bed (1.0 cm×4.08 cm/9.94 g) composed of 80:20 vol. % 20×40 US standard mesh size (30:1/SiO₂:Al₂O₃) H-ZSM-5 from Comparative Example 2: 20×40 US standard mesh size copper/KMnO₄ (Not Pre-Reduced) impregnated silica gel from Inventive Example 9. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

### NO REMOVAL EXAMPLE B

NOₓ test of a moisture unequilibrated stacked bed configured with a 1.0 cm×4.08 cm/10.01 g top layer composed of 80:20 vol. % 20×40 US standard mesh size (30:1/SiO₂:Al₂O₃) H-ZSM-5 from Example 2: 20×40 US standard mesh size copper/KMnO₄ (Not Pre-Reduced) impregnated silica gel from Inventive Example 9 and a 1.0 cm×4.08 cm/10.39 g bottom layer composed of 20×40 US standard mesh size ASZM-TEDA from Comparative Example 1. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

### NO REMOVAL EXAMPLE C

NOₓ test of a moisture unequilibrated stacked bed configured with a 1.0 cm×4.08 cm/9.64 g top layer composed of 80:20 vol. % 20×40 US standard mesh size (30:1/SiO₂:Al₂O₃) H-ZSM-5 from Comparative Example 2: 20×40 US standard mesh size copper/KMnO₄ (Reduced via heat treatment at 71° C.) impregnated silica gel from Inventive Example 9 and a 1.0 cm×4.08 cm/9.35 g bottom layer composed of 20×40 US standard mesh size ASZM-TEDA from Comparative Example 1. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

### NO REMOVAL EXAMPLE D

NOₓ test of a moisture unequilibrated bed (1.0 cm×4.08 cm/11.10 g) composed of 80:20 vol. % 20×40 US standard mesh size (30:1/SiO₂:Al₂O₃) H-ZSM-5 impregnated with 6 wt % HNO₃ from Comparative Example 5: 20×40 US standard mesh size copper/KMnO₄ (Not Pre-Reduced) impregnated silica gel from Inventive Example 9. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

### NO REMOVAL EXAMPLE E

NOₓ test of a moisture unequilibrated stacked bed configured with a 1.0 cm×4.08 cm/10.93 g top layer composed of 80:20 vol. % 25×40 US standard mesh size (30 :1/SiO₂:Al₂O₃) H-ZSM-5 with 5 wt % Bentonite and 6 wt % HNO₃ from Inventive Example 10:20×40 US standard mesh size copper/KMO₄ (Not Pre-Reduced) impregnated silica gel from Inventive Example 9 and a 1.0 cm×4.08 cm/9.35 g bottom layer composed of 20×40 US standard mesh size ASZM-TEDA from Comparative Example 1. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

### NO REMOVAL EXAMPLE F

NOₓ test of a moisture unequilibrated stacked bed configured with a 1.0 c×4.08 cm/11.02 g top layer composed of 80:20 vol. % 25×40 US standard mesh size (30:1/SiO₂:Al₂O₃) H-ZSM-5 with 5 wt % Bentonite and 6 wt % HNO₃ from Inventive Example 10:20×40 US standard mesh size copper/KMnO₄ (Pre-Reduced) impregnated silica gel from Inventive Example 11 and a 1.0 cm×4.08 cm/9.37 g bottom layer composed of 20×40 US standard mesh size ASZM-TEDA from Comparative Example 1. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

### NO REMOVAL EXAMPLE G

NOₓ test of a moisture unequilibrated stacked bed configured with a 1.0 cm×4.08 cm/11.02 g top layer composed of 70:30 vol. % 25×40 US standard mesh size (30:1/SiO₂:Al₂O₃) H-ZSM-5 with 5 wt % Bentonite and 6 wt % HNO₃ from Inventive Example 10:20×40 US standard mesh size copper/KMnO₄ (Pre-Reduced) impregnated silica gel from Inventive Example 11 and a 1.0 cm×4.08 cm/9.37 g bottom layer composed of 20×40 US standard mesh size ASZM-TEDA from Comparative Example 1. The test was conducted using an NO₂ feed concentration and relative humidity of 200 ppm and 15±2%, respectively.

The feed gas was introduced to the top of the bed at a flow rate of 5.22 SLPM resulting in a media velocity of ˜6.6 cm/sec.

### NO COMPARATIVE REMOVAL EXAMPLE H

NOₓ test of a moisture unequilibrated bed (1.0 cm×4.08 cm/10.50 g) composed of 20×40 US standard mesh size particles of commercially available ASZM-TEDA carbon from Comparative Example 1. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

### NO COMPARATIVE EXAMPLE I

NOₓ test of a moisture unequilibrated bed (1.0 cm×4.08 cm/9.92 g) composed of 20×40 US standard mesh size particles of commercially available (30:1/SiO₂:Al₂O₃) H-ZSM-5 from Comparative Example 2. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

### NO COMPARATIVE EXAMPLE J

NOₓ test of a moisture unequilibrated stacked bed configured with a 1.0 cm×4.08 cm/8.83 g top layer composed of 20×40 US standard mesh size particles of (30:1/SiO₂:Al₂O₃) H-ZSM-5 from Comparative Example 2 and a 1.0 cm×4.08 cm/10.38 g bottom layer composed of 20×40 US standard mesh size particles of ASZM-TEDA from Comparative Example 1. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

### NO COMPARATIVE EXAMPLE K

NO_{X} test of a moisture unequilibrated bed (1.0 cm×4.08 cm/10.22 g) composed of 80:20 vol. % 20×40 US standard mesh size (30:1/SiO₂:Al₂O₃) H-ZSM-5 from Comparative Example 2 : 20×40 US standard mesh size copper impregnated silica gel from Comparative Example 4. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

### NO COMPARATIVE EXAMPLE L

NOₓ test of a moisture unequilibrated stacked bed configured with a 1.0 cm×4.08 cm/10.11 g top layer composed of 80:20 vol. % 20×40 US standard mesh size (30:1/SiO₂:Al₂O₃) H-ZSM-5 from Comparative Example 2: 20×40 US standard mesh size copper impregnated silica gel from Comparative Example 4 and a 1.0 cm×4.08 cm/10.45 g bottom layer composed of 20×40 US standard mesh size ASZM-TEDA from Comparative Example 1. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

### NO COMPARATIVE EXAMPLE M

NOₓ test of a moisture unequilibrated bed (1.0 cm×4.08 cm/10.88 g) composed of 20×40 US standard mesh size (30:1/SiO₂:Al₂O₃) H-ZSM-5 impregnated with 6 wt % HNO₃ from Comparative Example 5. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

### NO COMPARATIVE EXAMPLE N

NOₓ test of a moisture unequilibrated stacked bed configured with a 1.0 cm×4.08 cm/10.51 g top layer composed of 20×40 US standard mesh size (30:1/SiO₂:Al₂O₃) H-ZSM-5 impregnated with 3 wt % HNO₃ from Comparative Example 6 and a 1.0 cm×4.08 cm/10.44 g bottom layer composed of 20×40 US standard mesh size ASZM-TEDA from Comparative Example 1. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

### NO COMPARATIVE EXAMPLE O

NOₓ test of a moisture unequilibrated stacked bed configured with a 1.0 cm×4.08 cm/12.30 g top layer composed of 80:20 vol. % 20×40 US standard mesh size (30:1/SiO₂:Al₂O₃) H-ZSM-5 impregnated with 6 wt % HNO₃ from Comparative Example 5: 20×40 US standard mesh size copper impregnated silica gel from Comparative Example 4 and a 1.0 cm×4.08 cm/10.39 g bottom layer composed of 20×40 US standard mesh size ASZM-TEDA from Comparative Example 1. The test was conducted using an NO₂ feed concentration and relative humidity of 200 ppm and 15±2%, respectively. The feed gas was introduced to the top of the bed at a flow rate of 5.22 SLPM resulting in a media velocity of ˜6.6 cm/sec.

The resultant measurements for NO breakthrough were as follows:

TABLE 2

| Example # | NO Breakthrough Time (min) |
|---|---|
| A | 9 |
| B | 23 |
| C | 15 |
| D | * |
| E | 33 |
| F | 21 |
| G | 28 |
| H | 13 |
| I | <1 |
| J | 8 |
| K | 1 |
| L | 9 |
| M | * |
| N | 14 |
| O | 13 |

* no breakthrough time registered

Thus, the inventive examples show a clear improvement over the comparative and non-oxidized species in terms of multiple threat gas removal. The pre-reduced products show good results as well, with greater reliability in terms of long-term stability. Thus such pre-reduced oxidizer-treated filter materials provide excellent filter capabilities with far reduced possibility of destabilization during storage and usage.

While the invention was described and disclosed in connection with certain preferred embodiments and practices, it is in no way intended to limit the invention to those specific embodiments, rather it is intended to cover equivalent structures structural equivalents and all alternative embodiments and modifications as may be defined by the scope of the appended claims and equivalents thereto.

What we claim is:

1. A filter medium comprising multivalent metal-doped silicon-based gel materials, wherein said materials exhibit a BET surface area of between than 100 and 600 m²/g; a pore volume of between about 0.18 cc/g to about 0.7 cc/g as measured by nitrogen porosimetry; a cumulative surface area measured for all pores having a size between 20 and 40 Å of between 50 and 150 m²/g; and wherein the multivalent metal doped on and within said silicon-based gel materials is present in an amount up to 25% by weight of the total amount of the silicon-based gel materials, wherein a pre-reduced oxidizing material has been contacted on the surface thereof of at least some of said silicon-based gel materials.

2. The filter medium of claim 1 wherein said BET surface area is between 150 m²/g and 400 m²/g; a pore volume of between about 0.25 to about 0.5 cc/g; a cumulative surface area measured for all pores having a size between 20 and 40A of between 80 and 120 m²/g; wherein said multivalent metal is present in an amount up to about 20%.

3. The filter medium of claim 1 wherein said multivalent metal is selected from the group consisting of cobalt, iron, manganese, zinc, aluminum, chromium, copper, tin, antimony, tungsten, indium, silver, gold, platinum, mercury, palladium, cadmium, nickel, and any combinations thereof.

4. The filter medium of claim 3 wherein said multivalent metal is copper.

5. The filter medium of claim 2 wherein the metal within said metal-doped silicon-based gel materials is selected from the group consisting of cobalt, iron, manganese, zinc, aluminum, chromium, copper, tin, antimony, indium, tungsten, silver, gold, platinum, mercury, palladium, cadmium, nickel, and any combinations thereof.

6. The filter medium of claim 5 wherein said multivalent metal is copper.

7. The filter medium of claim 1 wherein said pre-reduced oxidizing material is selected from at least one Class 1 oxidizing material, at least one Class 2 oxidizing material, at least one Class 3 oxidizing material, at least one Class 4 oxidizing material, and any mixtures thereof.

8. The filter medium of claim 7 wherein said pre-reduced oxidizing material is pre-reduced permanganate.

9. A filter system comprising the filter medium as defined in claim 1.

10. A filter system comprising the filter medium as defined in claim 2.

11. A filter system comprising the filter medium as defined in claim 3.

12. A filter system comprising the filter medium as defined in claim 4.

13. A filter system comprising the filter medium as defined in claim 5.

14. A filter system comprising the filter medium as defined in claim 6.

15. A filter system comprising the filter medium as defined in claim 7.

16. A filter system comprising the filter medium as defined in claim 8.

17. The filter system of claim 9 wherein said system includes a stacked bed of at least one other filter medium.

18. The filter system of claim 17 wherein said at least one other filter medium is selected from the group consisting of a carbon-based medium, a zeolite-based medium, a different silica-based medium, and any combinations thereof.

19. The filter system of claim 8 wherein said system includes a stacked bed of at least one other filter medium.

20. The filter system of claim 19 wherein said at least one other filter medium is selected from the group consisting of a carbon-based medium, a zeolite-based medium, a different silica-based medium, and any combinations thereof.
